(19) United States
(12) Patent Application Publication
(10) Pub. No.: US 2006/0149218 A1
(43) Pub. Date: Jul. 6, 2006

(54) Methods and apparatus for treating the interior of a blood vessel

(76) Inventors: Charles R. Slater, Fort Lauderdale, FL (US); Brett E. Naglreiter, Hollywood, FL (US); Scott L. Jahrmarkt, Miami Beach, FL (US); Thomas O. Bales, Coral Gables, FL (US); Banning G. Lary, Miami, FL (US)

Correspondence Address: GORDON & JACOBSON, P.C., 60 LONG RIDGE ROAD, SUITE 407, STAMFORD, CT, 06902, US

(21) Appl. No.: 10/544,082

(22) PCT Filed: Feb. 4, 2004
(86) PCT No.: PCT/US04/03249

§ 371 Date: Jul. 28, 2005

(30) Foreign Application Priority Data
Feb. 5, 2003 (US) 10358523

(51) Int. Cl. A61M 31/00

(52) U.S. Cl. 604/509; 604/523; 604/96.01

(57) ABSTRACT

Methods and apparatus for treating the interior of a blood vessel include a variety of catheter designs, methods and apparatus for occluding a blood vessel, methods and apparatus for locating an occlusion device, methods and apparatus for locating a treating device at the site of blood vessel tributaries, and methods and apparatus for dispensing treating agent.

This application is a continuation-in-part of application Ser. No. 09/898,867 filed Jul. 3, 2001, the complete disclosure of which is hereby incorporated by reference herein. This application is also related to co-pending application Ser. No. 10/328,085 filed Dec. 23, 2002, the complete disclosure of which is incorporated herein by reference.

### BACKGROUND OF THE INVENTION

1. Field of the Invention

The invention relates to the treatment and correction of venous insufficiency. More particularly the invention relates to a minimally invasive procedure using a catheter-based system to treat the interior of a blood vessel. The invention has particular application to varicose veins although it is not limited thereto.

2. State of the Art

Parent application Ser. No. 09/898,867 discloses an apparatus for delivering an intravascular drug such as a sclerosing agent (or a microfoam sclerosing agent) to a varicose vein. The apparatus includes a catheter having three concentric tubes. The innermost tube has a guide wire lumen and an inflation lumen. The distal end of the innermost tube has an integral inflatable occlusion balloon in fluid communication with the inflation lumen. The intermediate tube has a lumen through which the innermost tube extends. The distal end of the intermediate tube has a self-expanding balloon with a plurality of fluid pores in fluid communication with the intermediate tube lumen. The outer tube has a lumen through which the intermediate tube extends. Sclerosing agent is dispensed through the intermediate tube to pores located at the distal end of the intermediate tube or in the self-expanding balloon. Veins are sclerosed as the self-expanding balloon is pulled through and ultimately out of the vein.

While particular methods and apparatus were disclosed in the parent application for occluding the blood vessel, dispensing sclerosing agent, and locating tributaries, it will be appreciated that it would be desirable to have additional manners of accomplishing the same.

### SUMMARY OF THE INVENTION

It is therefore an object of the invention to provide methods and apparatus for the minimally invasive treatment of varicose veins wherein only minimal anaesthesia is required.

It is another object of the invention to provide methods and apparatus for the minimally invasive treatment of varicose veins wherein tributary veins are treated simultaneously with the vein to which they connect.

It is an additional object of the invention to provide methods and apparatus for the minimally invasive treatment of varicose veins and connecting tributaries wherein the entire wall of the vein is evenly sclerosed.

Another object of the invention is to provide methods and apparatus for the minimally invasive treatment of varicose veins which do not utilize high concentration sclerosing agents.

Yet another object of the invention is to provide methods and apparatus for the minimally invasive treatment of varicose veins which do not require that the practitioner carefully monitor the duration, rate, or progression of treatment.

Still another object of the invention is to improve upon the methods and apparatus of the previously incorporated parent application.

It is also an object of the invention to provide methods and apparatus for treating the interior of a blood vessel.

It is also an object of the invention to provide methods and apparatus for occluding a blood vessel prior to treatment.

It is another object of the invention to provide methods and apparatus for locating an occlusion device in a blood vessel.

It is an additional object of the invention to provide methods and apparatus for locating tributaries of a treated blood vessel.

It is a further object of the invention to provide catheter arrangements for treating the interior of a blood vessel.

It is another object of the invention to provide methods and apparatus for the minimally invasive treatment of varicose veins.

In accord with these objects which will be discussed in detail below, an apparatus according to the present invention includes a catheter device having three concentric tubes: an inner tube, an outer tube, and an intermediate tube. Each tube has a proximal end and a distal end with a lumen extending therethrough. As used herein, the term proximal means closest to the practitioner and the term distal means farthest from the practitioner when the apparatus is in use. An inflatable balloon is located at or near the distal end of inner tube and a fluid valve is coupled to the proximal end of the inner tube. The balloon is inflated by injecting fluid through the valve and is held in an inflated condition by closing the valve. A fluid outlet is located at or near the distal end of the intermediate tube and a “plunger” is coupled to the proximal end of the intermediate tube. The plunger is movable within the outer tube defining a fluid reservoir of varying size between the proximal end of the outer tube and the plunger. The plunger permits fluid communication between the fluid reservoir and the lumen of the intermediate tube. The proximal end of the outer tube is provided with a trifurcated fitting including a Tuohy-Borst type connector. The proximal end of the inner tube extends through the Tuohy-Borst connector which provides a fluid seal between the inner tube and the outer tube and which locks the inner tube in position relative to the outer tube. A pullwire is coupled to the plunger and extends through a central port of the trifurcated fitting which maintains a fluid seal between the pullwire and the outer tube. The third port of the trifurcated fitting is provided with a female Luer with a check valve which permits one-way fluid access into the fluid reservoir. According to the presently preferred embodiment, the distal end of the inner tube is provided with a radiopaque tip and a safety wire extends within the inner tube providing the inner tube with stiffness and maneuverability for precise placement of the inflatable balloon. The wire is bonded to or captures the entire device, thereby helping to keep it together. Further according to the presently preferred embodiment, the outer tube is transparent and provided with a plurality of movable exterior markers which are useful in performing the methods of the invention.

According to alternate embodiments of the apparatus, other types-of tracking devices may be used at the tip of the inner tube rather than the radiopaque tip. Examples of such devices include an LED or an illuminated fiber optic which is visible through the skin, or a magnet which can be detected with an electromagnetic sensor.

Methods of the invention include examining the patient and marking the patient's leg to indicate the entry site, the occlusion site and important sites (e.g. tributaries) along the blood vessel. The distal end of the outer tube is placed adjacent to the entry site and the inner tube and intermediate tube are extended outside the patient along the leg to the occlusion site. The intermediate tube is then drawn back from the occlusion site to the first important site marking proximal of the occlusion site. One of the movable exterior markers on the outer tube is then moved to the position occupied by the plunger. The intermediate tube is then moved to the next proximal important site marking on the leg and another marker on the outer tuber is moved to the corresponding position of the plunger. These steps are repeated until all of the important site markings have been recorded with the movable markers on the outer tube. The catheter is then reset so that the distal ends of the inner tube and intermediate tube are adjacent to each other. A 10cc-20cc syringe is loaded with sclerosing agent and is attached to the female luer. While holding the catheter in an upward direction, 10cc of sclerosing agent is injected into the fluid reservoir and the intermediate tube until a few drops exit the fluid outlet of the intermediate tube and the tubes are purged of air bubbles. If necessary, the syringe is reloaded with additional sclerosing agent.

The inner and intermediate tubes are then inserted through a hemostasis valve or cut-down into the blood vessel and maneuvered through the vessel until the distal end of the outer tube abuts the vessel or hemostasis valve. The balloon is then inflated using a 3cc-5cc syringe coupled to the proximal end of the inner tube. Infusion of sclerosing agent is commenced by pulling the pullwire so that the plunger is moved proximally forcing fluid out of the fluid reservoir through the intermediate tube and out of the fluid outlets at the distal end of the intermediate tube. When the plunger reaches one of the markers on the outer tube, additional sclerosing agent may be injected using the 10cc-20cc syringe. The plunger is then moved to the next marker and additional sclerosing agent is injected. After all of the markers have been passed by the plunger, the balloon is deflated and the catheter device is removed from the patient.

Further in accord with these objects which will be discussed in detail below, the occlusion devices of the present invention include: sponges, umbrellas, chemical sealants, ligation, and a suction device. The umbrella designs may incorporate elastic or superelastic struts, a tubular inflatable cuff, or a wire hoop with a basket.

The methods for locating the occlusion device according to the invention include: ultrasound, palpation, fluoroscopic and magnetic resonance imaging, placing a bright light (e.g. LED) at the end of the occlusion device, pressure monitoring, and a technique similar to the placement of a “wedge catheter”.

The methods for locating tributaries are of two types: one involves pre-marking on the patient's skin, and the other does not use marking. The pre-marking methods include locating the tributaries via ultrasound, transillumination, or other type of imaging, and marking the patient's skin at the locations of the tributaries. After pre-marking several additional methods can be used. One method involves marking the treating device by placing the treating device on the patient's skin and marking it in locations that align with the marks on the patient's skin. A second method following pre-marking involves using a bright light at the tip of the drug delivery device. A third method following pre-marking involves using ultrasound to locate the tip of the drug delivery device. A fourth method following pre-marking involves using palpation to locate the tip of the drug delivery device. A fifth method following pre-marking involves using a magnet at the tip of the drug delivery device and a magnetic follower on the patient's skin. Several different types of magnetic followers are provided.

The methods for locating tributaries without pre-marking include: ultrasound imaging during the procedure, placing a light source at the tip of the drug delivery device bright enough to illuminate the tributaries through the patient's skin, external illumination with or without an image intensifying system, real time fluoroscopy or other type of imaging, and pressure gradient detection.

Further embodiments of catheter-based treating devices include: a catheter having an atraumatic floppy guide wire tip attached to the distal end of an inflatable occlusion balloon, a dual monorail catheter system, a two-way single monorail catheter system, a two-way clip-on catheter system, and a multi-perforated catheter which does not move during drug delivery.

Additional objects and advantages of the invention will become apparent to those skilled in the art upon reference to the detailed description taken in conjunction with the provided figures.

### BRIEF DESCRIPTION OF THE DRAWINGS

FIG. 1 is a schematic side elevational view of a first catheter device according to the invention with the inner and intermediate tubes withdrawn;

FIG. 2 is a schematic side elevational view of the first catheter device according to the invention with the inner and intermediate tubes extended;

FIG. 3 is a schematic side elevational view of the first catheter device according to the invention in use;

FIGS. 4a-4e are schematic illustrations of the distal ends of the inner tube and intermediate tube of the first catheter device during use;

FIG. 5 is a schematic view of a sponge occlusion device in a state of partial deployment;

FIG. 6 is a schematic view of a first umbrella occlusion device in a state of partial deployment;

FIG. 7 is a schematic view of a second umbrella occlusion device in a state of partial deployment;

FIG. 8 is a schematic view of a third umbrella occlusion device in a state of partial deployment;

FIG. 9 is a schematic view illustrating deployment of a tissue sealant at an occlusion site;

FIG. 10 is a schematic view of a compression device at an occlusion site;

FIG. 11 is a schematic view illustrating venous ligation as an occlusion method;

FIG. 12 is a schematic view of a suction apparatus for occluding a blood vessel;

FIG. 13 is a schematic view illustrating the positioning of an occlusion device with the use of ultrasound;

FIG. 14 is a schematic view illustrating the positioning of an occlusion device with the use of palpation;

FIG. 15 is a schematic view illustrating the positioning of an occlusion device with the use of fluoroscopic imaging;

FIG. 16 is a schematic view illustrating the positioning of an occlusion device with the use of a bright light coupled to the occlusion device;

FIG. 17 is a schematic view illustrating the positioning of an occlusion device with the use of a pressure monitor;

FIG. 18 is a schematic view illustrating the positioning of an occlusion device with the use of a wedge placement technique;

FIG. 19 is a schematic view illustrating the pre-marking of a patient's leg indicating the locations of tributaries;

FIG. 20 is a schematic view illustrating the marking of a treatment device using the pre-marked leg as a guide;

FIG. 21 is a schematic view illustrating the location of a treatment device at a tributary using a first embodiment of a magnetic follower and the pre-markings on the patient's leg;

FIG. 22 is a schematic view illustrating the location of a treatment device at a tributary using a second embodiment of a magnetic follower and the pre-markings on the patient's leg;

FIG. 23 is a schematic view illustrating the location of a treatment device at a tributary using a third embodiment of a magnetic follower and the pre-markings on the patient's leg;

FIG. 24 is a schematic view illustrating the location of a treatment device at a tributary using a fourth embodiment of a magnetic follower and the pre-markings on the patient's leg;

FIG. 25 is a schematic view illustrating the location of a treatment device at a tributary using external IR illumination;

FIG. 26 is a schematic illustration of a catheter having an atraumatic floppy guide wire tip attached to the distal end of an inflatable occlusion balloon;

FIG. 27 is a schematic illustration of a dual monorail catheter system;

FIG. 28 is a schematic illustration of a single monorail catheter system;

FIG. 28A is a section taken along line A-A in FIG. 24;

FIG. 29 is a schematic illustration of a clip-on catheter system;

FIG. 29A is a section taken along line A-A in FIG. 25;

FIG. 30 is a schematic illustration of another embodiment of the invention which utilizes a multi-perforated catheter which does not move during drug delivery;

FIG. 31 is a schematic illustration of a multi-perforated weeping catheter;

FIG. 32 is a schematic illustration of a second embodiment of a multi-perforated weeping catheter;

FIG. 33 is a schematic perspective view of a portion of a third embodiment of a multi-perforated weeping catheter;

FIG. 34 is a longitudinal cross sectional view of a fourth embodiment of a multi-perforated weeping catheter;

FIG. 35 is a perspective view of the distal end of a fifth embodiment of a multi-perforated weeping catheter;

FIG. 36 is a side elevational view of the distal end of the fifth embodiment of a multi-perforated weeping catheter with its occlusion balloon inflated; and

FIG. 37 is a section taken along line 37-37 in FIG. 36.

### DETAILED DESCRIPTION OF THE PREFERRED EMBODIMENTS

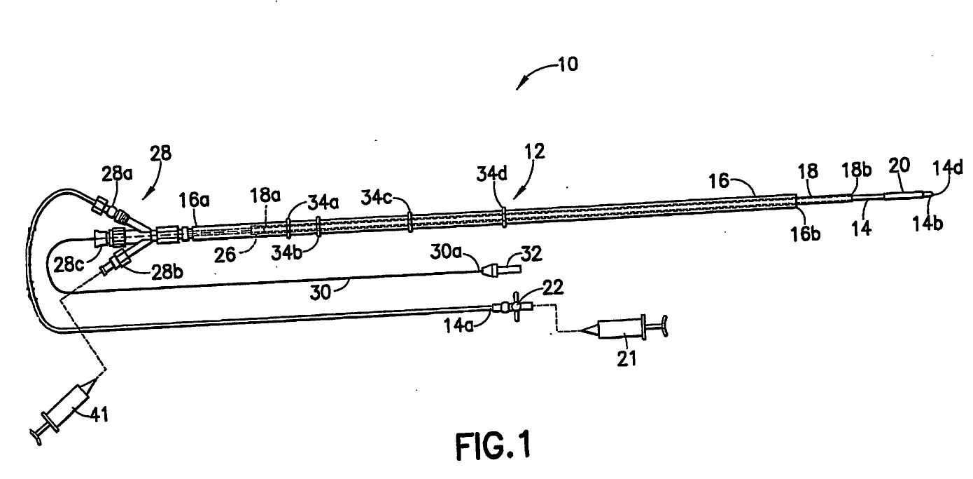

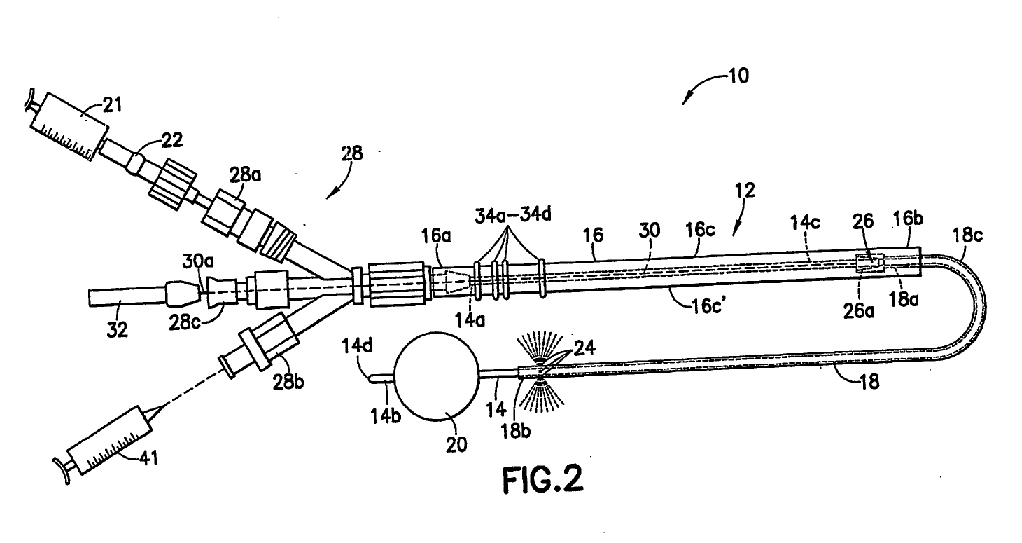

Referring now to FIGS. 1 and 2, an apparatus 10 according to the present invention includes a catheter device 12 having three concentric tubes: an inner tube 14, an outer tube 16, and an intermediate tube 18. Each tube 14, 16, 18 has a proximal end 14a, 16a, 18a and a distal end 14b, 16b, 18b with a lumen 14c, 16c, 18c extending therethrough. As used herein, the term proximal means closest to the practitioner and the term distal means farthest from the practitioner when the apparatus is in use.

An inflatable balloon 20 is located at or near the distal end 14b of inner tube 14 and a fluid valve 22 is coupled to the proximal end 14a of the inner tube 14. The balloon 20 is inflated by injecting fluid (e.g. saline) through the valve 22 and is held in an inflated condition by closing the valve 22.

As seen best in FIG. 2, one or more fluid outlet(s) 24 are located at or near the distal end 18b of the intermediate tube 18 and a “plunger” 26 is coupled to the proximal end 18a of the intermediate tube 18. According to the presently preferred embodiment, the fluid outlets 24 include a plurality of radial outlets and a fluid seal (not shown) closes the annular space between the tube 14 and the tube 18 at a location distal of the outlets 24. The fluid seal (not shown) is heat formed and makes a sliding (dynamic) seal. The plunger 26 is movable within the outer tube 16 defining a fluid reservoir 16c′ of varying size between the proximal end 16a of the outer tube 16 and the plunger 26. For example, FIGS. 1 and 2 illustrate two extreme locations of the plunger 26, FIG. 1 showing a small reservoir and FIG. 2 showing a large reservoir. The plunger 26 permits fluid communication between the fluid reservoir 16c′ and the lumen 18c of the intermediate tube 18. According to the presently preferred embodiment, the plunger 26 is provided with an indication 26a as seen best in FIG. 2. According to the presently preferred embodiment, the indication 26a is a sealing O-ring contrasting in color to that of the plunger 26.

The proximal end 16a of the outer tube 16 is provided with a trifurcated fitting 28 including a Tuohy-Borst type connector 28a, a female Luer 28b with check valve (not shown) and a Luer 28c housing a seal connector (not shown).

The proximal end 14a of the inner tube 14 extends through the Tuohy-Borst connector 28a which provides a fluid seal between the inner tube 14 and the outer tube 16 and which selectively locks the inner tube 14 in position relative to the outer tube 16.

The female Luer 28b with check valve permits one-way fluid access into the fluid reservoir 16c′ of the outer tube 16.

A pullwire 30 is coupled to the plunger 26 and extends through the luer 28c of the trifurcated fitting 28 which maintains a fluid seal between the pullwire 30 and the outer tube 16. The proximal end 30a of the pullwire 30 is provided with a handle 32. According to the presently preferred embodiment, the handle is a striking color (e.g. orange) so that it can be quickly located.

According to the presently preferred embodiment, the distal end 14b of the inner tube 14 is provided with a radiopaque tip 14d and a safety wire (not shown in FIGS. 1 or 2) extends within the inner tube 14 providing the inner tube with stiffness and maneuverability for precise placement of the inflatable balloon.

Further according to the presently preferred embodiment, the outer tube 16 is transparent and provided with a plurality of movable exterior markers 34a-34d which are used in conjunction with the indication 26a on the plunger 26 in performing the methods of the invention described in more detail below. The presently preferred markers are elastic O-rings.

According to alternate embodiments of the apparatus, other types of tracking devices may be used at the distal end of the inner tube rather than the radiopaque tip. Examples of such devices include an LED or an illuminated fiber optic which is visible through the skin, or a magnet which can be detected with an electromagnetic or magnetic sensor or follower.

The apparatus 10 is intended for use with and thus also preferably includes two syringes, a 3-5cc syringe 21 for inflating the balloon and a 10-20cc syringe 41 for injecting sclerosing agent.

Although it is not necessary to perform the procedure in an operating room, it is considered prudent for the initial examination to be performed in an out-patient suite in a hospital or in an operating room in the event that any unforeseen events occur that may require surgical intervention.

The patient should first be examined under ultrasound, palpation, fluoroscopy or other means for venous valve insufficiency and varicose veins. If the physician determines that the patient is a candidate for closure of the saphenous vein as a means of eliminating the varicosities, the patient will be admitted for the procedure.

Preferably, a photograph of the patient's leg is taken both before and after the procedure so that the results of the procedure can be readily ascertained.

The patient is preferably sedated with a mild sedative such as Percocet, or the like, one hour prior to the procedure. An IV line may be inserted in the patient's arm and vital signs monitored throughout the procedure.

While the patient is standing, the saphenofemoral junction is located using Doppler or other ultrasonic techniques and the skin marked over this junction with a washable marker. Similarly, the saphenous vein and its major tributary junctions is traced using ultrasound and its path marked on the surface of the skin with a marker.

If varicosities are present above the knee only, then the length of the saphenous vein from the knee to the groin will be treated either through a cut down to the saphenous vein or by a percutaneous stick into the saphenous vein (or both) using a catheter sheath introducer. If the disease is prevalent below the knee, then a similar incision or percutaneous stick will be made in the saphenous vein at the level of the ankle and the vein sclerosed from the ankle to the knee. If the disease is prevalent in both the upper and lower leg, then an incision or percutaneous stick will be made in the saphenous vein at the level of the ankle and the vein sclerosed from the ankle to the groin.

The patient lies down with his/her leg elevated 30 to 45 degrees to allow blood to drain from the leg. The patient's leg is scrubbed with a standard surgical preparation medium, such as betadine and the site prepared for an aseptic procedure. Lidocaine or other local anesthetic is injected into the area around the vein with a small needle.

Prior to use, the apparatus 10 should be examined to determine that it is functioning properly. This should include sliding the plunger in and out through the outer tube and dilating the balloon with 3cc of sterile saline.

The following procedure assumes that the patient's skin has been previously marked with the entry site, the occlusion site and important sites (e.g. tributaries) along the vessel. It also assumes that the catheter device can be laid down on the patient's leg while maintaining sterility.

With the inner tube 14 and the intermediate tube 18 drawn into the outer tube 16 as shown in FIG. 1, the distal end 16b of the outer tube 16 is located at the entry site (Oust proximal to the hemostasis valve of the CSI). While the outer tube 16 is maintained in position, the inner tube 14 and the intermediate tube 18 are pulled out of the outer tube 16, by grasping and pulling the intermediate tube, until the balloon 20 is over the mark on the skin representing the occlusion site.

The inner tube 14 is locked in position by tightening the Tuohy Borst valve 28a. Locking the Tuohy Borst valve assures that when the apparatus is inserted into the leg, the balloon will inflate at the desired occlusion site. It also assures that the balloon will not migrate backwards when the sclerosing agent is dispensed.

Starting with the distal end 18b of the intermediate tube 18 abutting the balloon 20, the pullwire 30 is pulled such that the intermediate tube moves backwards until the fluid outlet 24 is located at the next marking on the patient's leg (e.g. a tributary site). With the apparatus in this position, the closest marker (o-ring) 34d is moved over the tube 16 until it is aligned with the indicia 26a on the plunger 26. The pullwire 30 is pulled again and this step is repeated for each of the marks on the patient's leg, using the O-rings 34c, 34b, 34a to mark the corresponding location of the plunger 26. It will be appreciated that the number of markers shown in the Figures is arbitrary and more or fewer markers may be provided.

After all of the desired markers 34a-34d have been placed along the tube 16, the intermediate tube 18 is pulled distally until its distal end 18b abuts the balloon 20 as shown in FIG. 2.

As mentioned above, two syringes are used to operate the apparatus, a 3-5cc syringe 21 to expand the balloon and a 10-20cc syringe 41 to dispense the sclerosing agent. The smaller syringe is filled with sterile saline and attached to the fluid valve 22 (a Luer with a stop cock). The larger syringe is filled with sclerosing agent and attached to the female Luer 28b. While holding the intermediate tube 18 in an upward direction, 10cc of the sclerosing agent is injected through the check valve 28b into the reservoir 16c′ of the tube 16, through the plunger 26, and up through the tube 18 such that a few drops of fluid emerge from the fluid outlets 24 on the distal end of the tube 18. The physician should ensure that the tubes 16, 18 are purged of air bubbles. If necessary, the larger syringe is reloaded with additional sclerosing agent before proceeding.

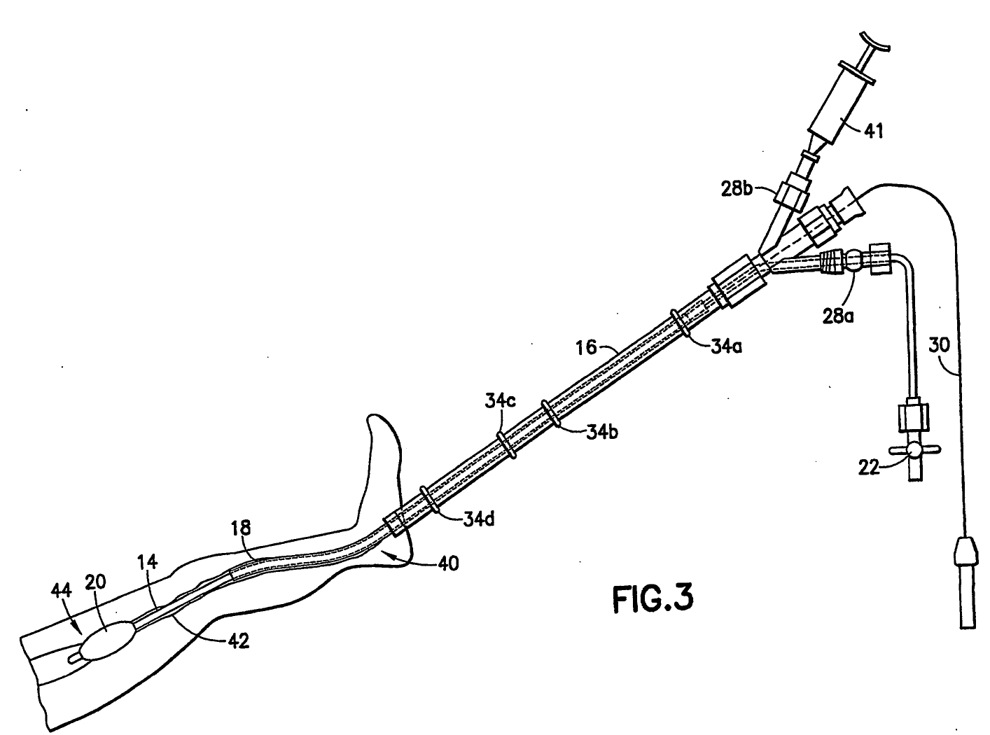

The inner tube 14 and the intermediate tube 18 are then inserted into a percutaneous stick 40 in the saphenous vein 42 as shown in FIG. 3. The tubes 14, 18 are maneuvered to the occlusion location 44 preferably with the aid of the tip indicator 14d of the tube 14. As mentioned above, the tip indicator 14d may be radiopaque and thus located with fluoroscopy. Alternatively, the tip 14d may be provided with an LED or an optical fiber which causes it to glow bright enough to be seen through the skin. Still alternatively, the tip 14d may be magnetic and thus located with electromagnetic or magnetic equipment.

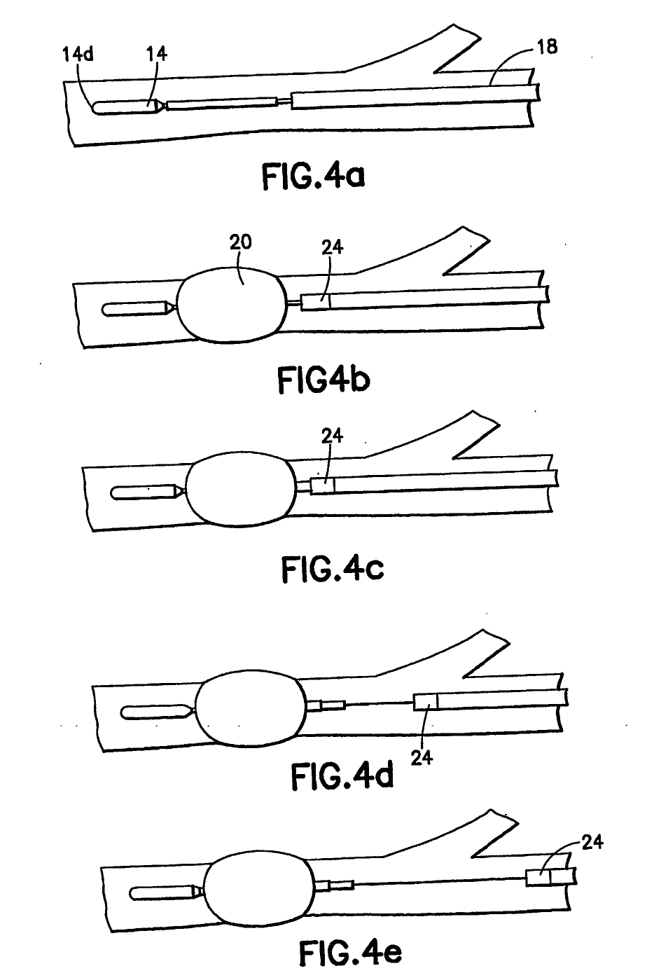

With the apparatus in position as shown in FIG. 4a, the balloon 20 is expanded with the small syringe as shown in FIG. 4b. According to the presently preferred embodiment, preferably no more than 5cc should be injected into the balloon which will expand to a diameter of approximately 21 mm upon injection of 5cc. Table 1 illustrates a typical relationship between the injection volume and the balloon diameter.

TABLE 1

| Injection volume ±0.1 cc | Balloon Diameter ±1 mm |
|---|---|
| 1 | 12 |
| 2 | 15 |
| 3 | 18 |
| 4 | 19 |
| 5 | 21 |

The balloon is preferably inflated slowly with sterile saline or radiopaque media until it totally occludes the vessel. Ultrasound, fluoroscopy, palpation, tugging, etc. can be used to ensure that the balloon is adequately inflated. Once the balloon is inflated, the stopcock 22 is closed by rotating the stopcock 90°.

The infusion procedure is begun by pulling the pullwire 30 back until the O-ring on the piston lines up with the first O-ring marker previously located on the tube 16. Pulling on the pullwise causes the plunger 26 to be moved toward the proximal end of the tube 16, which in turn forces the sclerosing agent out of the fluid outlets 24 in the distal end of the tube 18 which is also moved away from the balloon 20 as shown in FIG. 4c. This releases a controlled and evenly distributed amount of sclerosing agent which is well suited for sclerosing a vein with no tributaries. When the end of the tube 18 reaches a tributary, as shown in FIG. 4d and as indicated by the placement of the O-rings 34a-34d, it is desirable to release additional sclerosing agent to contract the tributary as well as the vein. This may be accomplished by injecting additional sclerosing agent with the large syringe which remains attached to the injection port 28b. After the additional sclerosing agent is released, movement of the tube 18 is resumed as shown in FIG. 4e.

Injection of this bolus of sclerosing agent may be directed and facilitated with a fork-like device (not shown) that compresses the outside of the leg on either side of the fluid outlets 24. A roller may also be used to force the sclerosing agent up the tributary. This process is repeated for other large tributaries. Preferably no more than 20cc of 1.5% sclerosing agent should be used in this procedure.

When the tube 18 is fully withdrawn, the balloon 20 is deflated by aspiration and the tube 14 is removed from the vein. The entry site may be sutured before dressing. However, according to the presently preferred embodiment, the size of the introducer is only 6-French which may produce a sufficiently small wound so as not to require suturing. However, the leg is preferably immediately wrapped in a gauze-type dressing (e.g., KERLIX® available from Kendall Co., Walpole, Mass.). A length of foam rubber padding is preferably placed over the gauze and over the saphenous vein that was sclerosed. An elastic bandage (e.g., COACH® or ACE®) is preferably placed over the foam rubber to keep it in place. An additional elastic bandage may be placed over the first elastic bandage to ensure that the vein remains compressed and that blood does not flow back into the treated veins.

The patient should be advised to rest with his/her leg elevated for approximately 30 minutes. The patient can then walk to the car, elevate the leg in the car and then keep the leg elevated in bed overnight. Occasional flexure of the foot, ankle and leg should be encouraged. It is preferred that the patient be re-examined the following day. The dressings should then be replaced and the patient instructed on how to self apply new dressings and bandages. The dressings, foam pads and bandages may be kept in place for five to seven days. After five to seven days, the patient should be reexamined and, if indicated, the dressings and foam removed. The compression bandage should be worn for an additional week.

The patient should be asked to return for follow-up at one month and three months if indicated. The patient may also be asked to return at one year to evaluate the long term effectiveness of the procedure.

The benefits of the methods and apparatus of the invention include:

(i) Sclerosing agents are painless in the vascular system as compared to laser or RF ablation that can be extremely painful.

(ii) The occlusion balloon prevents the sclerosing agent from entering the deep venous system via the saphenofemoral or saphenopopliteal junctions.

(iii) The catheter is 6-Fr in diameter and is easily maneuvered through the vein.

(iv) Only one injection of anesthesia is required at the puncture site, resulting in less pain and toxicity to the patient.

(v) Venous access via a small cut down or by use of a catheter sheath introducer produces a very minimal scar, resulting in a better cosmetic impact.

(vi) The recovery time is faster with fewer cosmetic complications as compared to stripping.

(vii) Tributaries can be treated as well as the main veins resulting in a better cosmetic impact.

(viii) Veins below the knee can be treated.

(ix) The total procedural time is greatly reduced.

(x) The apparatus is less expensive than laser and RF apparatus.

(xi) The procedure is performed in an outpatient setting.

(xii) The apparatus automatically assures that the correct amount of sclerosing agent is evenly distributed without requiring the practitioner to carefully monitor the duration of treatment.

FIGS. 5-12 illustrate additional occlusion methods and devices according to the present invention.

FIG. 5 illustrates a catheter 110 located within a blood vessel 1. A sponge 112, coupled to a guide wire 114 extending through the catheter 110, is released from the distal end of the catheter 110 by pushing the guide wire distally or by withdrawing the catheter proximally. When the procedure is complete, pulling the guide wire 114 (or pushing the catheter) retrieves the sponge into the catheter whereupon the catheter may be withdrawn.

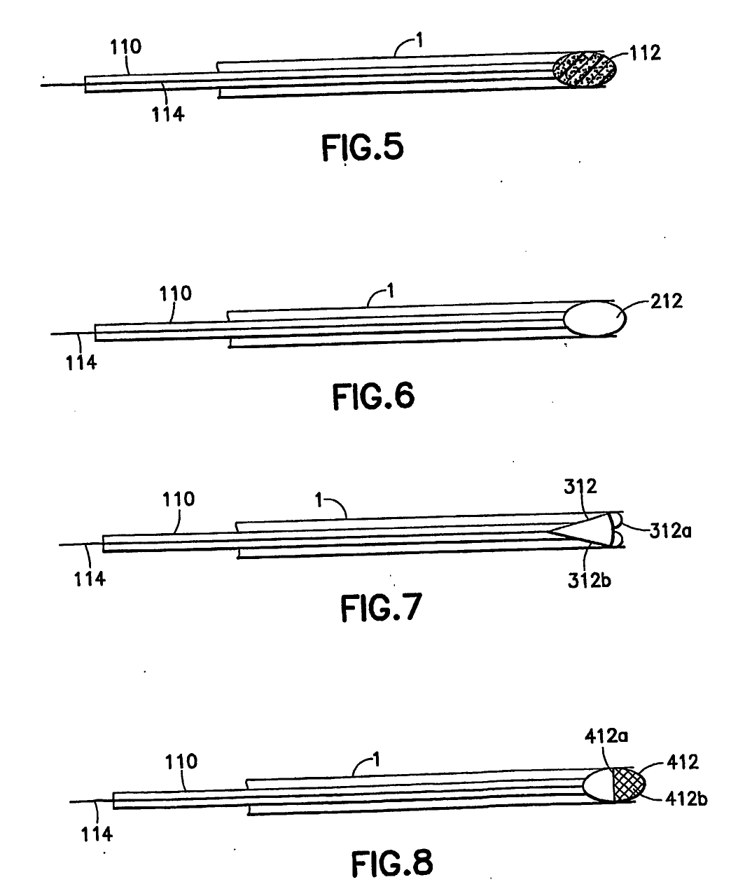

FIG. 6 illustrates a catheter 110 located within a blood vessel 1. A first umbrella occlusion device 212, coupled to a guide wire 114 extending through the catheter 110, is released from the distal end of the catheter 110 by pushing the guide wire distally (or by withdrawing the catheter proximally). When the procedure is complete, pulling the guide wire 114 (or pushing the catheter) retrieves the umbrella 212 into the catheter whereupon the catheter may be withdrawn.

The umbrella 212 is a structure made of elastic or superelastic wires or struts which are biased to be in an “open,” larger-diameter configuration when there is no external restraint on them, e.g. when released from the catheter. These struts or wires are covered with a membrane or very fine mesh which effectively occludes the flow of blood. Alternatively, the struts can be biased to the closed position, and the structure may be expanded by applying a force to compress the structure axially (by means of two push-pull wires) so as to expand it. (See the previously incorporated co-pending application Ser. No. 10/328,085).

FIG. 7 illustrates a catheter 110 located within a blood vessel 1. A second umbrella occlusion device 312, coupled to a guide wire 114 extending through the catheter 110, is released from the distal end of the catheter 110 by pushing the guide wire distally or by pulling the catheter proximally. When the procedure is complete, pulling the guide wire 114 or pushing the catheter distally retrieves the umbrella 312 into the catheter whereupon the catheter may be withdrawn.

The umbrella 312 includes a tubular inflatable cuff 312a at the distal end of a funnel-shaped membrane 312b. When inflated, the tubular cuff assumes a toroidal shape which expands the membrane to the form of a funnel, contacts the inside wall of the blood vessel and occludes fluid flow.

FIG. 8 illustrates a catheter 110 located within a blood vessel 1. A third umbrella occlusion device 412, coupled to a guide wire 114 extending through the catheter 110, is released from the distal end of the catheter 110 by pushing the guide wire distally or pulling the catheter proximally. When the procedure is complete, pulling the guide wire 114 or pushing the catheter retrieves the umbrella 412 into the catheter whereupon the catheter may be withdrawn.

The umbrella 412 includes an expandable loop of wire 412a coupled to an impervious membrane or film bag 412b. Once extended, the loop and bag expand to fill the lumen of the blood vessel, blocking the flow of fluid.

FIG. 9 illustrates a method of occluding a blood vessel 1 by delivering a glue/sealant 116 from a source 118 via a catheter 110 to the site of occlusion. Suitable sealants include butyl-cyanoacrylate, fibrin solution, and other tissue-sealing materials. The sealant is used in a liquid or semi-liquid form to prevent it from embolizing. Once the sealant is applied, the closing of the vein may be assisted by externally applied pressure.

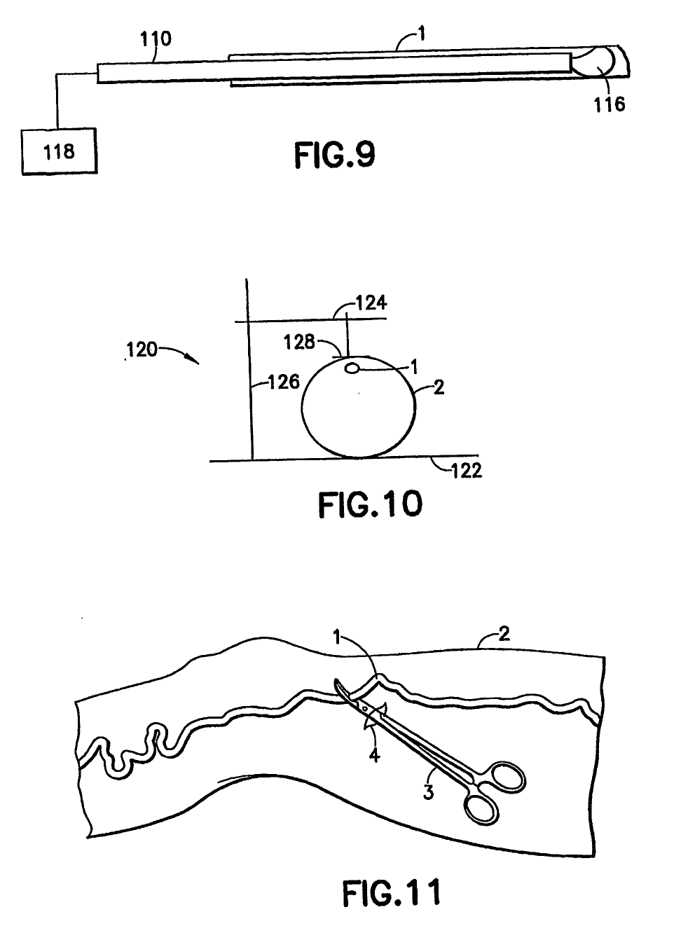

FIG. 10 illustrates in schematic form an apparatus for applying external pressure to a blood vessel 1 in a patient's leg 2. The apparatus 120 generally includes a lower member 122 which is located beneath the patient's leg 2, an upper member 124 located above the patient's leg and coupled to the lower member by a vertical post 126. The upper member 124 may be provided with a pressure pad 128 located directly above the blood vessel 1. Similar apparatus are known for use in closing arterial puncture sites at the groin following arterial access in angioplasty procedures, for example.

It is possible to occlude the superficial saphenous vein solely by the application of external compression by means of a mechanical assistive device. Examples of compression devices include: inflatable cuffs, inflatable cuffs with means for localizing compression (for example, a rubber bougie or ball), and a mechanical clamping device with a padded “foot.”

FIG. 11 illustrates another method of occluding a blood vessel 1 in a patient's leg 2 with the use of a surgical clamp 3 delivered to the occlusion site via an incision 4. In lieu of a clamp, the practitioner may occlude the blood vessel with a suture (not shown).

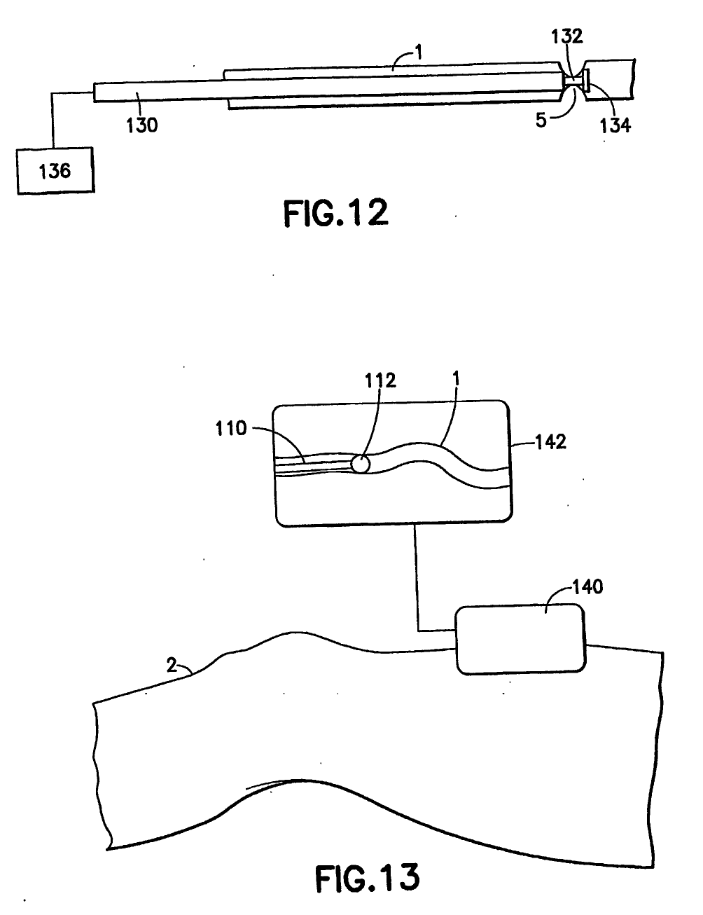

FIG. 12 illustrates a suction device for occluding a blood vessel 1. The suction device includes a catheter 130 having a coaxial extension 132 and a disk 134 which define an annulus at the end of the catheter. The catheter 130 is coupled to a vacuum source 136 and the wall of the blood vessel 1 is drawn into the annulus as illustrated at 5 in FIG. 12.

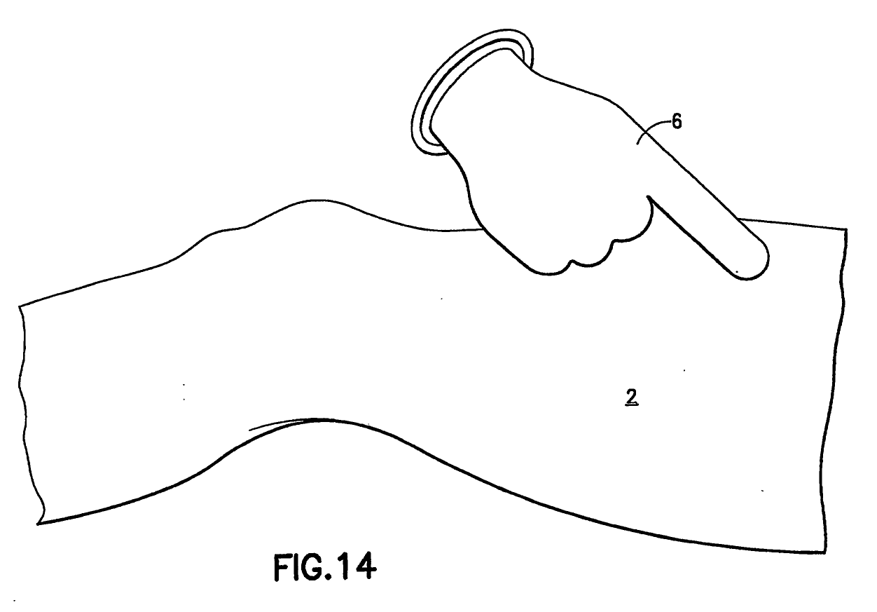

FIGS. 13-18 illustrate methods and apparatus for locating an occlusion device in a blood vessel. Turning now to FIG. 13, an ultrasound device 140 having a display 142 is used to locate a vein 1 in a patient's leg 2. The ultrasound device will also display the location of a catheter 110 and occlusion device 112 within the vein 1.

FIG. 14 illustrates a method of locating an occlusion device via palpation. A skilled practitioner can determine the desired location of the occlusion balloon by examination of the leg. The distal end of the occlusion catheter can then be located by palpation, especially if there is a distal bulb or other feature on the occlusion catheter. FIG. 14 shows the practitioner's hand 6 palpating the patient's leg 2.

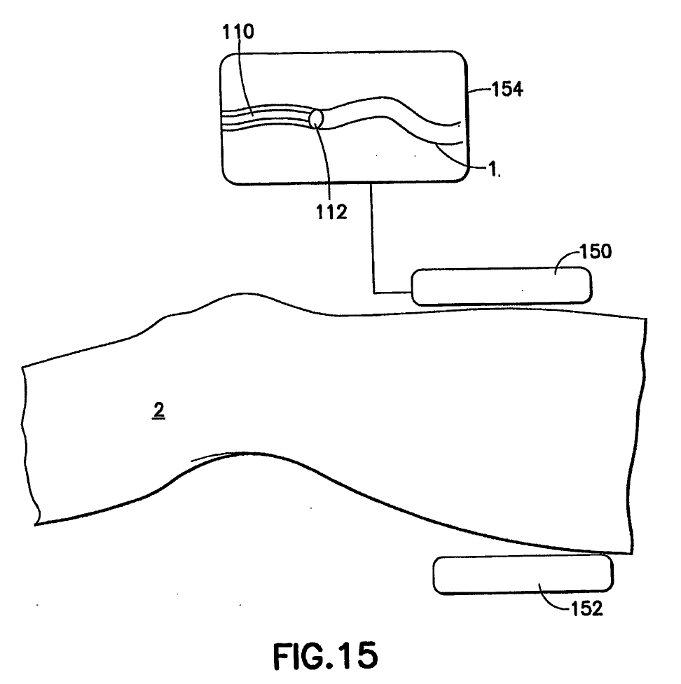

FIG. 15 illustrates a method of locating an occlusion device using imaging such as fluoroscopy or magnetic resonance imaging (MRI). In fluoroscopy, a detector 150 is placed over the patient's leg 2 and a source of radiation 152 is placed beneath the leg 2. The detector 150 is coupled to a display 154 which illustrates the patient's blood vessel 1, the catheter 110 and the occlusion device 112. In MRI, a means for varying a magnetic field 152 is provided and may be placed beneath the leg, around the leg, or otherwise as is known in the art. A radio-frequency detecting circuit or system 150 is placed over the leg, around the leg, or otherwise as is known in the art, and is coupled to the display 154 which illustrates the blood vessel 1.

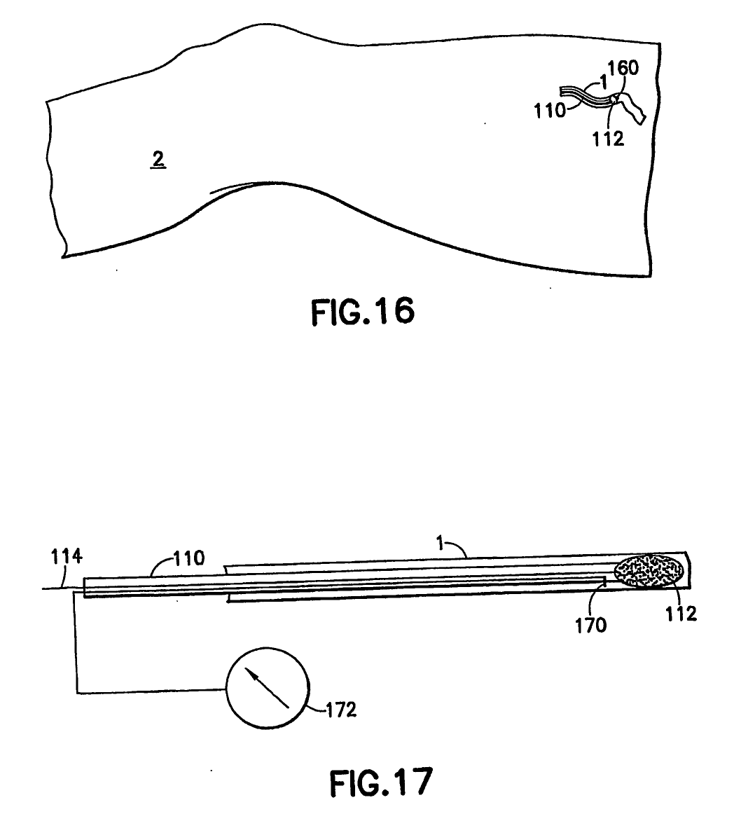

FIG. 16 illustrates a catheter 110 having an occlusion device 112 and a light source 160 (e.g., an LED or fiber optic tip) adjacent the occlusion device 112. Once the desired location of the occlusion balloon in vein 1 has been determined by examining the leg 2, the occlusion device is easily located by the light emitted from the light source 160 which is bright enough to be seen through the patient's skin.

FIG. 17 illustrates a catheter 110 with an occlusion device 112 located within a blood vessel 1. The catheter 110 is provided with a pressure sensor 170 which is coupled to a pressure gauge 172. The pressure in the femoral vein is lower than the pressure in the saphenous vein. Therefore, by monitoring the fluid pressure at the distal end of the occlusion catheter 110, it is possible to determine when the pressure sensor moves from the saphenous vein into the junction of the saphenous vein and the femoral vein. If actuation of the occlusion device in the saphenous vein is desired, the pressure sensor can then be withdrawn proximally into the saphenous vein proximal of the femoral vein (as indicated by a sensed increase in blood pressure), and the occlusion device actuated therein.

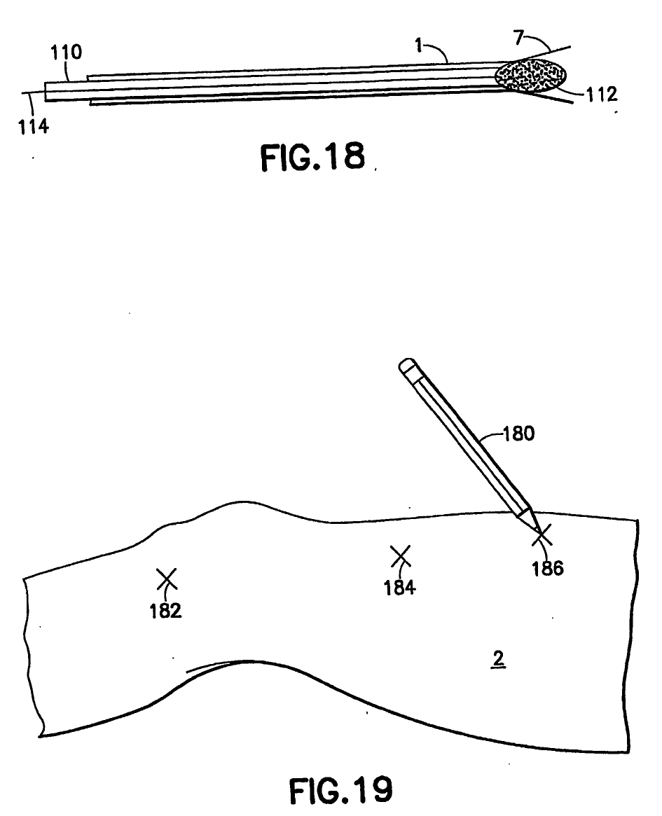

FIG. 18 shows a catheter 10 having an occlusion device 112 coupled to deployment means 114. According to a method of the invention, the occlusion device is located at the desired site by first passing it into the femoral vein 7, then deploying it, then pulling it back until it “wedges” against the junction of the saphenous vein 1. In the case of an occlusion balloon, by deflating the balloon, withdrawing it a short distance (1-2 cm), and re-inflating it, the occlusion balloon can be correctly located at the desired location in the saphenous vein.

According to the methods of the invention, an additional bolus of treating agent is optionally dispensed when the treating catheter passes a tributary blood vessel. FIGS. 19-24 illustrate methods for locating tributary blood vessels which include pre-marking the patient's skin. FIG. 19 shows the first step in which a marker 180 is used to make fiducial marks 182, 184, 186 on the surface of the leg 2 in registration with the side branches of the saphenous vein. These marks are made prior to the procedure of treating the blood vessels with the aid of ultrasound or other imaging (e.g., x-ray, MRI, or trans-illumination). Once these marks have been placed, the position of the catheter can be controlled by any of the following methods.

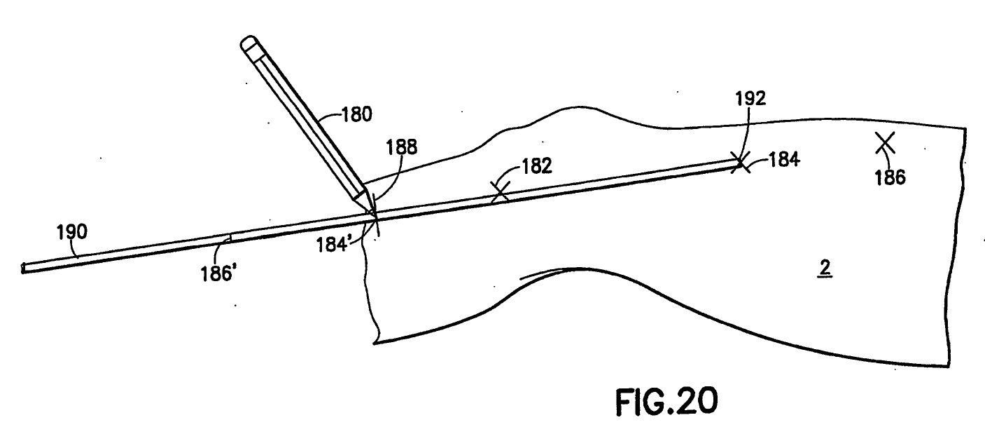

FIG. 20 shows a treating catheter 190 placed on top of the patient's leg 2. Fiducial marks are placed on the catheter by aligning the catheter on the outside of the leg along the path of the saphenous vein. The treating end 192 of the catheter 190 is positioned at each of the side-branch marks 186, 184, 182, etc. A corresponding fiducial mark 186′, 184′, etc. is placed on the catheter where the catheter will exit the venipuncture 188. In this way, the practitioner creates on the outside of the catheter an array of fiducial marks such that during the procedure whenever one of these marks is coincident with the venipuncture (or any other convenient index mark), the distal end 192 of the treating catheter 190 is adjacent to one of the side branches. Alternatively, in embodiments where a “pull wire” is used to retract the catheter, these marks can be applied to the pull wire.

Another (unillustrated) method of utilizing the pre-markings on the patient's leg is to use a catheter with a light source at its treating end such as the light source shown in FIG. 16. When the light source is seen under the side branch mark, additional treating agent is optionally dispensed. Still another (unillustrated) method of utilizing the pre-markings on the patient's leg is to palpate the location of the treating end of the catheter such as shown in FIG. 14. In this method, the treating catheter is preferably provided with a bulb or bougie which can be felt through the patient's skin. Thus, when palpation at or adjacent to pre-markings indicates location of the distal end of the catheter thereat, additional treating agent is optionally dispensed to treat the tributary blood vessel(s).

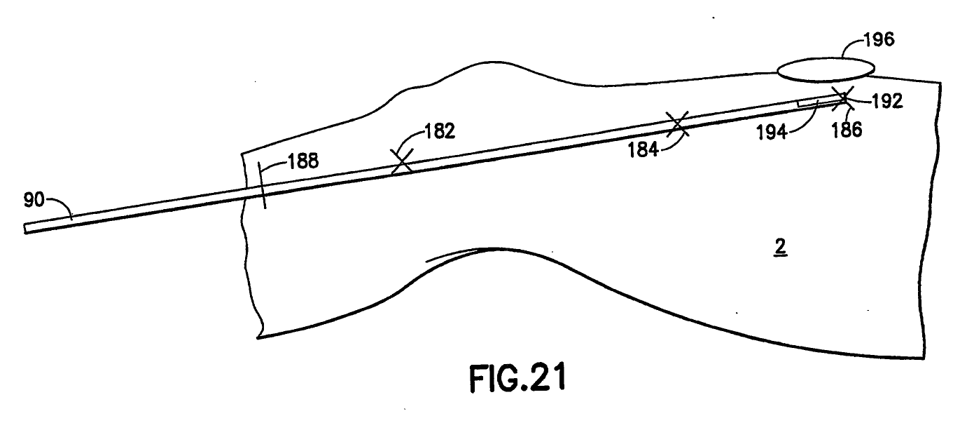

FIG. 21 illustrates a first method of utilizing the pre-markings on the patient's leg with a magnetic follower. Here the treating catheter 190 is provided with a magnet 194 at its treating end 192. A magnetic follower 196 is placed on the patient's leg 2. The follower rolls or slides along the surface of the leg showing the location of the treating end of the catheter. Whenever the follower passes over a pre-marking, additional treating agent is optionally dispensed to treat the tributary blood vessel(s).

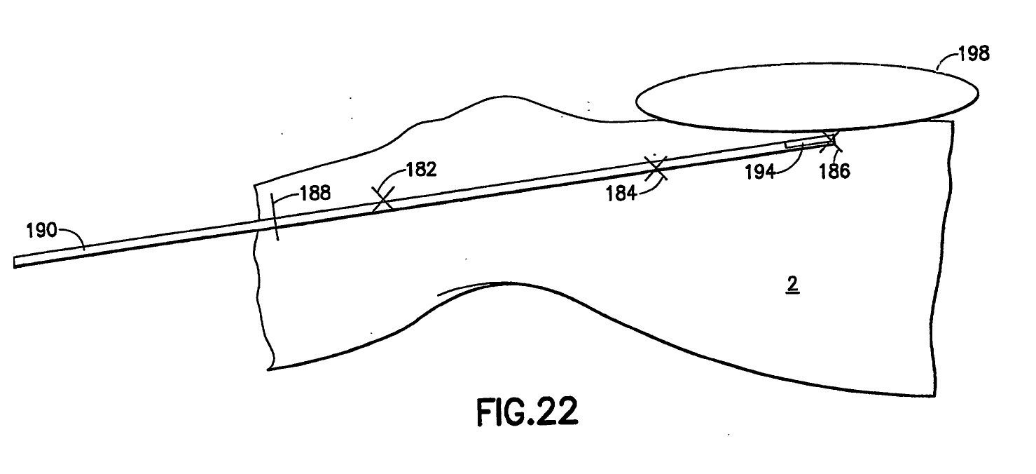

FIG. 22 illustrates another method of utilizing the pre-markings on the patient's leg with a magnet located on the treating end of a catheter. This method uses a transparent magnetic visualization screen 198 which contains iron filings. The screen is held over the markings on the patient's leg and when the magnet 194 on the catheter 190 passes under the screen, the iron filings show its movement. Whenever the screen indicates that the treating end of the catheter is located at a pre-marking, additional treating agent is optionally dispensed to treat the tributary blood vessel(s).

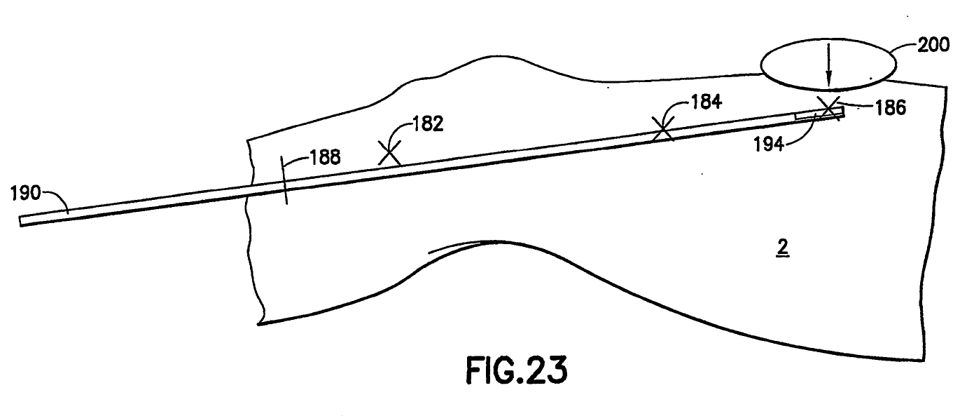

FIG. 23 illustrates another method of utilizing the pre-markings on the patient's leg with a magnet located on the treating end of a catheter. This method uses a hand held magnet detector such as a compass 200. The compass 200 is placed by the markings on the patient's leg and the compass needle indicates the passage of the magnet 194 on the treating end of the catheter 190. Whenever the magnet passes under a pre-marking, additional treating agent is optionally dispensed to treat the tributary blood vessel(s).

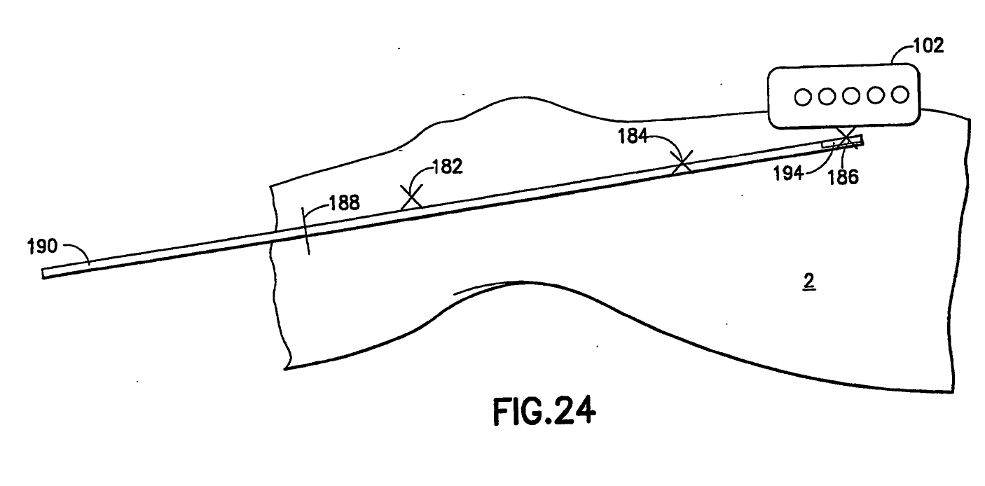

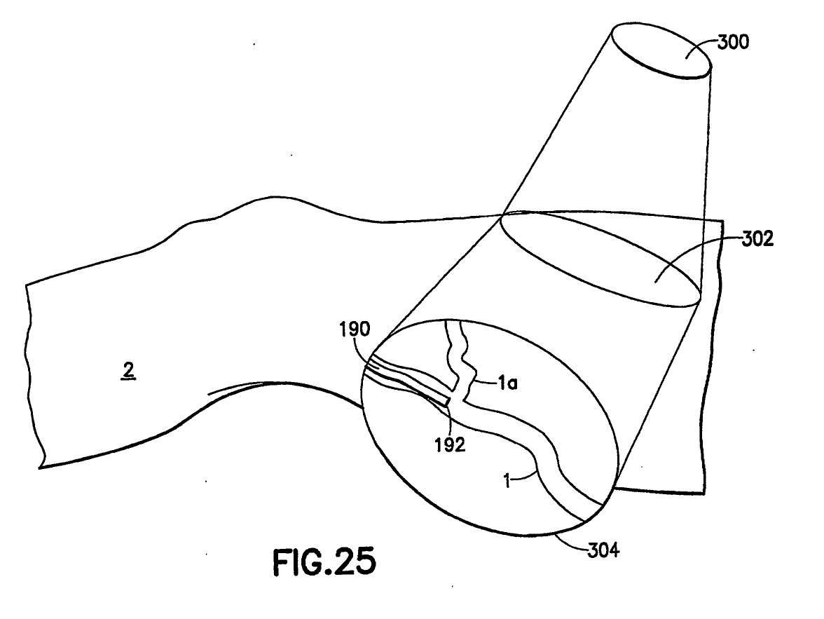

FIG. 24 illustrates another method of utilizing the pre-markings on the patient's leg with a magnet located on the treating end of a catheter. This method uses a hand held magnet detector such as an electronic device 202 having a plurality of ILDs which light as a magnet passes. The device 202 is placed by the markings on the patient's leg and the LEDs indicate the passage of the magnet 194 on the treating end of the catheter 190. Whenever the LED device indicates that the magnet is located under a pre-marking, additional treating agent is optionally dispensed to treat the tributary blood vessel(s).

The invention also contemplates methods of locating the treating end of a catheter at tributaries without pre-marking via different types of imaging such as ultrasound such as described above with reference to FIG. 13, fluoroscopic imaging such as described above with reference to FIG. 15, and a bright light coupled to the treating end of the catheter such as described above with reference to FIG. 16.

FIG. 25 illustrates the use of an external light source 300 which is used to direct light onto a region 302 of the patient's leg 2. The light source 300 is preferably an infrared (IR) light source, and an IR viewing device 304 (such as IR goggles) is used to determine the location of the treating end 192 of the catheter 90 at tributaries 1a in vein 1.

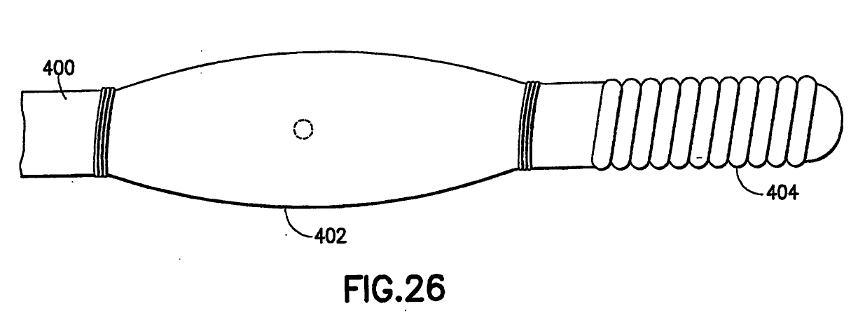

FIGS. 26-32 illustrate various catheter devices according to the invention.

Turning now to FIG. 26, an occlusion catheter 400 has an inflatable balloon 402 coupled to its distal end and an atraumatic floppy guide wire tip 404 coupled to the distal end of the balloon.

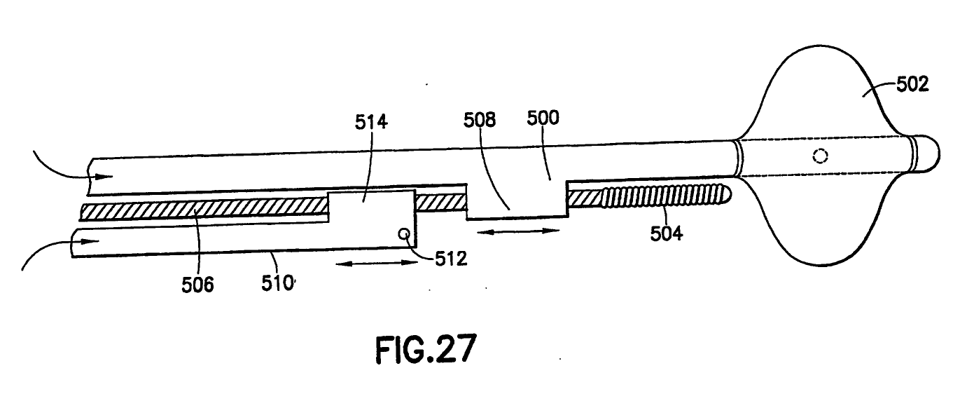

FIG. 27 illustrates a dual monorail system which includes an occlusion catheter 500 having an inflatable balloon 502 and a first monorail coupling 508. A guide wire 506 having an atraumatic tip 504 is arranged to pass through the monorail coupling 508. A treating catheter 510 having a distal fluid outlet 512 is also provided with a monorail coupling 514 through which the guide wire 506 also passes. From the foregoing, those skilled in the art will appreciate that the assembly is configured as shown but with the balloon 502 deflated. The guide wire is delivered to the site where the occlusion balloon is to be inflated. The occlusion catheter and treating catheter are delivered over the guide wire until the balloon is at the desired location. The balloon is then inflated. Treating fluid is then dispensed as the catheter 510 is withdrawn over the guide wire. At the locations of tributaries, additional treating fluid is optionally dispensed.

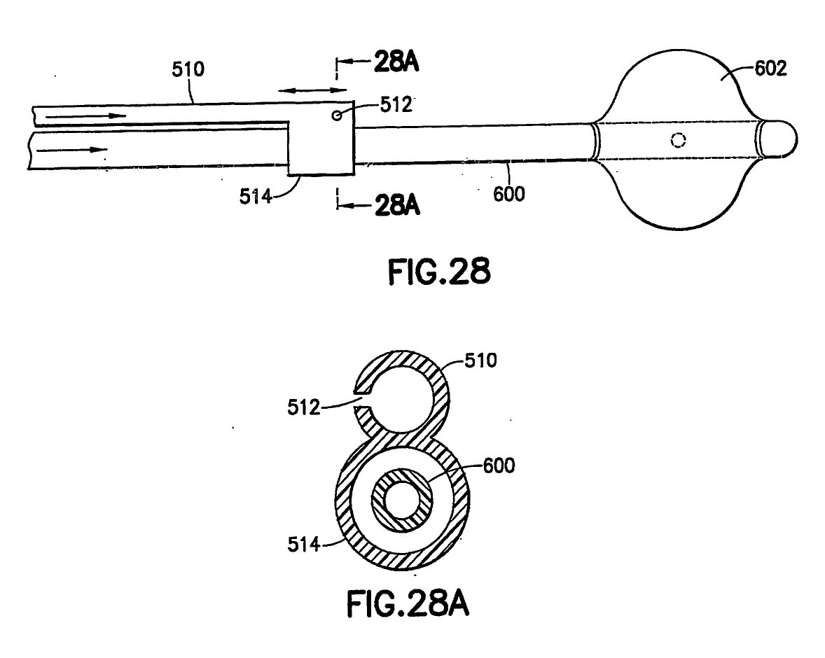

FIGS. 28 and 28A illustrate a single monorail system which includes an occlusion catheter 600 having an inflatable balloon 602 at its distal end and a treating catheter 510. The treating catheter 510 has a drug dispensing port 512 and a monorail coupling 514 through which the occlusion catheter 600 extends. From the foregoing, those skilled in the art will appreciate that the assembly is configured as shown but with the balloon 602 deflated. The occlusion catheter 600 and the treating catheter 510 are delivered through the blood vessel until the balloon is at the desired location. The balloon is then inflated. Treating fluid is then dispensed as the catheter 510 is withdrawn over the catheter 600. At the locations of tributaries, additional treating fluid is optionally dispensed. Alternatively, if desired, the treating catheter may be advanced over the occlusion catheter after occlusion is effected by the balloon. After fully advancing, the treating catheter may then be withdrawn and fluid dispensed to perform the therapy.

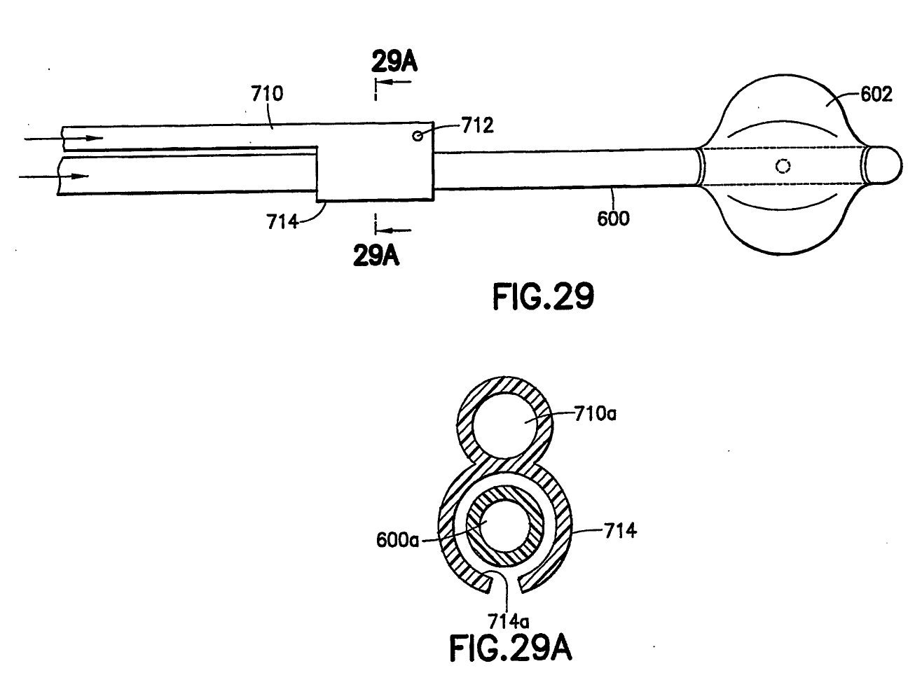

FIGS. 29 and 29A illustrate a clip-on monorail system which includes an occlusion catheter 600 having an inflatable balloon 602 at its distal end and a treating catheter 710. The treating catheter 710 has a drug dispensing port 712 and a clip-on monorail coupling 714 through which the occlusion catheter 600 extends. From the foregoing, those skilled in the art will appreciate that the assembly is configured as shown but with the balloon 602 deflated. The occlusion catheter 600 and the treating catheter 810 are delivered through the blood vessel until the balloon is at the desired location. The balloon is then inflated. Treating fluid is then dispensed as the catheter 710 is withdrawn over the catheter 600. At the locations of tributaries, additional treating fluid is optionally dispensed. Alternatively, if desired, the treating catheter 710 may be advanced over the occlusion catheter 600 after occlusion is effected by the balloon 602. After fully advancing, the treating catheter may then be withdrawn and fluid dispensed to perform the therapy.

FIG. 29A illustrates the inflation lumen 600a of the occlusion catheter 600, and the drug delivery lumen 710a of the treating catheter 700. The inner surface 714a of the clip-on monorail coupling 714 is preferably a lubricous contact surface.

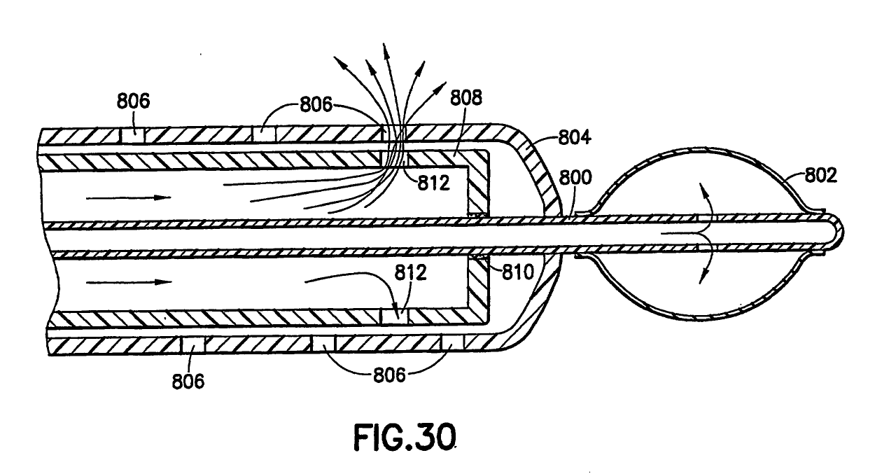

FIG. 30 illustrates an occlusion and drug delivery system which includes an occlusion catheter 800 having an inflatable balloon 802 at its end. A first coaxial outer catheter 804 extends over the occlusion catheter 800 and is preferably coupled to it. The catheter 804 has a plurality of perforations 806 along its length. A second coaxial inner catheter 808 extends over and is movable along the occlusion catheter 800 within the first coaxial outer catheter 804. The second coaxial inner catheter 808 is preferably provided with an annular fluid seal 810. The second coaxial inner catheter 808 is provided with at least one radial fluid outlet 812 which aligns with the perforations 806 in the first coaxial outer catheter 804 as the catheter 808 is moved along the catheter 800. From the foregoing, those skilled in the art will appreciate that the assembly is configured as shown but with the balloon 802 deflated. The three catheters are delivered through the blood vessel until the balloon is at the desired location. The balloon is then inflated. Treating fluid is then dispensed as the coaxial inner catheter 808 is withdrawn over the catheter 800 but with the coaxial catheter 804 in place.

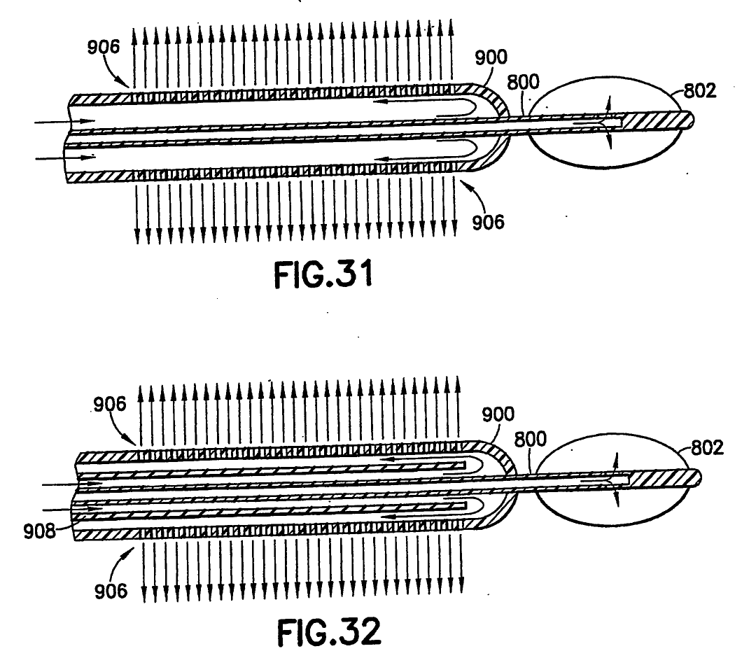

FIG. 31 illustrates an occlusion and drug delivery system which includes an occlusion catheter 800 having an inflatable balloon 802 at its end. A coaxial outer catheter 900 extends over the occlusion catheter 800 and is preferably coupled to it. The catheter 900 has a plurality of very small perforations 906 along its length. In use, the catheters are delivered through the blood vessel until the balloon is at the desired location. The balloon is then inflated. Treating fluid is then dispensed into the annular space between the catheters as shown by the arrows in FIG. 31. As the annular space fills, sufficient pressure is reached so that the fluid weeps out of the small perforations 906 along the length of the catheter 900.

FIG. 32 illustrates a second embodiment of a weeping catheter system. This arrangement is similar to the arrangement shown in FIG. 31 but for the addition of an annular baffle 908 between the catheter 800 and the catheter 900. The baffle prevents release of treating fluid through the perforations 906 until the fluid has first reached the distal end of the catheter system and then is redirected proximally in an annular space defined by the baffle 1008 and the weeping catheter 900.

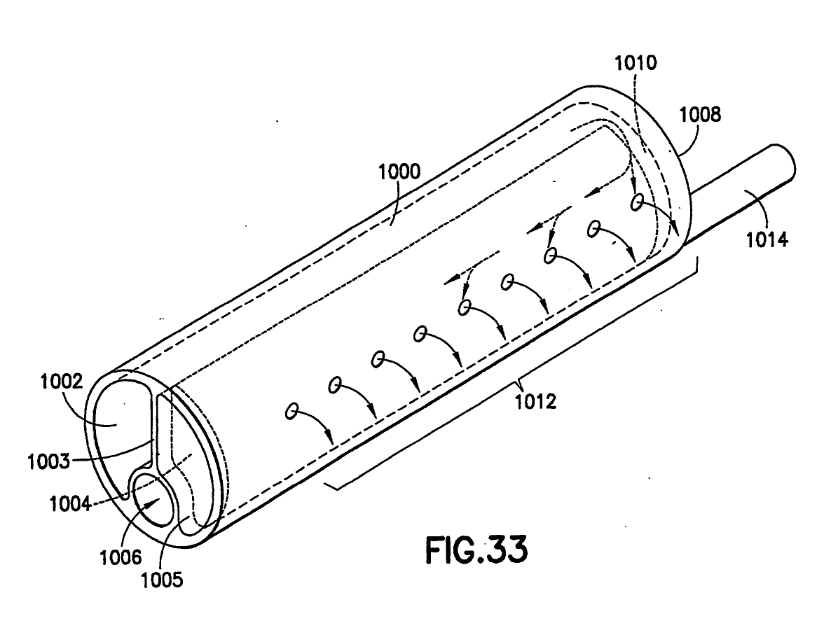

FIG. 33 illustrates a portion of a third embodiment of a weeping catheter 1000. The catheter has three lumens 1002, 1004, and 1006. The lumens 1002 and 1004 are larger than the lumen 1006 and are separated by a wall 1003. The ends of the lumens 1002 and 1004 are closed at 1008, but wall 1003 is stopped proximal of wall 1008 such that a fluid passage 1010 is formed to couple distal portions of the lumena 1002 and 1004. A plurality of perforations 1012 are provided along the length of the catheter 1000 in fluid communication with the lumen 1004. The proximal portion 1005 of the lumen 1004 is sealed. As shown in FIG. 33, a tubular extension 1014 is provided at the distal end of the catheter. This extension 1014 is in fluid communication with the lumen 1006 and is used to inflate a balloon not shown in this Figure. From the foregoing, those skilled in the art will appreciate that treating fluid delivered through lumen 1002 will travel to the end of the catheter and pass through the passage 1010 into the lumen 1004 where it will travel proximally past all of the perforations 1012 weeping out of the catheter.

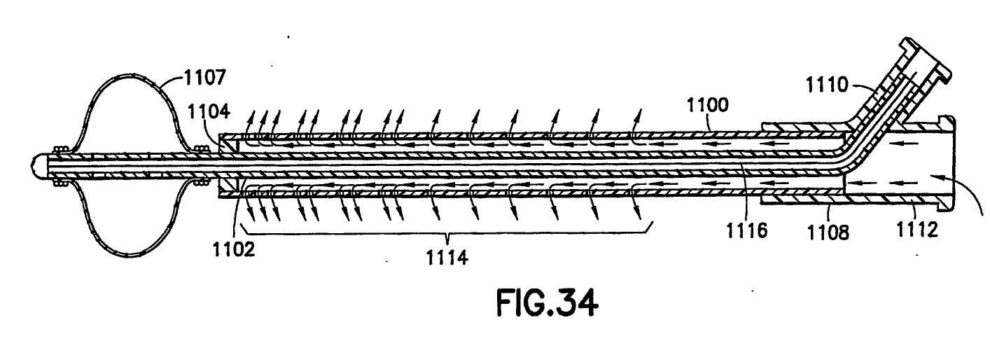

FIG. 34 shows a weeping catheter 1100 with a coaxial balloon inflation catheter 1102. The distal end of the catheter 1100 is provided with an annular seal 1104 between it and the inflation catheter 1102. The distal end of the inflation catheter 1102 is provided with an inflatable balloon 1107. The proximal end of the weeping catheter 1100 is coupled to a fluid coupling port 1108 having a side port 1110 and a main port 1112. The proximal end of the inflation catheter 1102 is coupled to the side port 1110. The weeping catheter 1100 has a plurality of perforations 1114 along at least a portion of its length. Preferably, a support wire 1116 is disposed inside the inflation catheter 1102 from its proximal end to its distal end to provide desired stiffness and tensile strength. From the foregoing, those skilled in the art will appreciate that fluid dispensed through the side port 1110 will inflate the balloon 1107 and treating fluid dispensed through the main port 1112 will weep through the perforations 1114.

According to the invention, the weeping catheters described above with reference to FIGS. 30-34 may be provided with different perforation configurations. The diameters of the perforations may be constant or variable. Preferred perforation sizes range from 0.002 inches to 0.007 inches, although perforations as small as 0.001 inches can be utilized. The spacing of the perforations may be constant or variable. Perforations may be provided in groups which are evenly spaced or variably spaced. The number of perforations per group may be constant or variable. These different configurations are chosen so as to provide either equal or biased infusion along the treating length of the weeping catheter. According to one embodiment, the perforations are dimensioned to prevent the passage of treating fluid until a predetermined fluid pressure is reached. According to another embodiment, the function of the perforations is achieved with a microporous material rather than discrete holes.

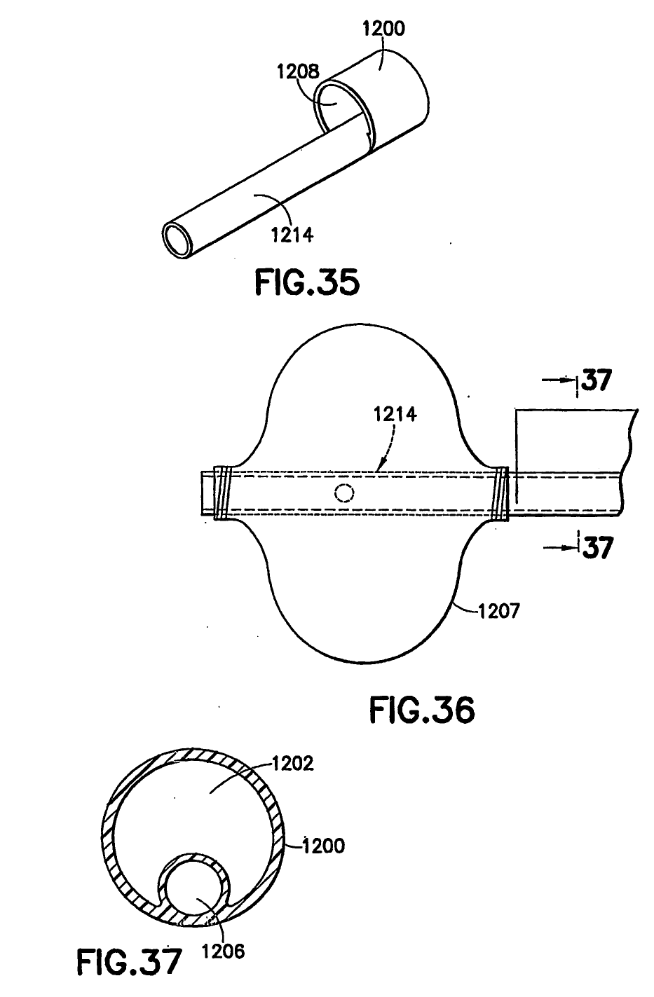

FIGS. 35-37 illustrate a portion of a weeping catheter 1200 which can be considered to be a combination of the catheters 1000 and 1100. In this embodiment, the inflation catheter (or lumen) 1206 is not coaxial with the weeping catheter 1200 and the infusion space (or lumen) 1202 is not annular as in the catheter 1100. However, the distal end 1214 of the inflation catheter is provided with an inflatable balloon 1207 which is substantially similar to the arrangement shown in FIG. 34. The distal end 1208 of the infusion space 1202 is sealed and a plurality of perforations into the infusion space are provided along the treating length of the catheter 1200 as described above with reference to the other weeping catheter embodiments, but not shown in FIGS. 35-37.

There have been described and illustrated herein several embodiments of methods and apparatus for treating the interior of a blood vessel. While particular embodiments of the invention have been described, it is not intended that the invention be limited thereto, as it is intended that the invention be as broad in scope as the art will allow and that the specification be read likewise. Thus, it will be appreciated that the methods and apparatus of the invention may be used in different combinations. It will therefore be appreciated by those skilled in the art that yet other modifications could be made to the provided invention without deviating from its spirit and scope as so claimed.

1-30. (canceled)

31. An apparatus for treating the interior of a blood vessel, comprising:
a first catheter having a proximal end and a distal end with a plurality of spaced apart perforations therebetween;
an occlusion device coupled to the distal end of said first catheter;
means for deploying the occlusion device; and
means for dispensing a treating agent through said perforations proximal of said occlusion device.

32. The apparatus according to claim 31, further comprising
a second catheter movable within said first catheter, said second catheter having at least one perforation adapted to align sequentially with said plurality of spaced apart perforations as said second catheter is moved through said first catheter, said second catheter adapted to receive a treating agent and dispense the treating agent through said at least one perforation.

33. The apparatus according to claim 32, further comprising:
an atraumatic tip coupled to the occlusion device.

34. The apparatus according to claim 32, wherein:
said occlusion device is one of a balloon, a sponge, an umbrella, and a sealant dispenser.

35. The apparatus according to claim 34, wherein:
said occlusion device is an inflatable balloon.

36. The apparatus according to claim 31, wherein:
said perforations are dimensioned to prevent passage of treating fluid until a predetermined fluid pressure is reached.

37. The apparatus according to claim 31, further comprising:
a coaxial cylindrical baffle located within said first catheter and defining an annular space between it and said first catheter.

38. The apparatus according to claim 31, further comprising:
an inflation catheter substantially coaxial with and extending through said first catheter,
wherein
said occlusion device is an inflatable balloon coupled to said inflation catheter.

39. The apparatus according to claim 31, wherein:
said means for deploying includes a first lumen in said first catheter, and
said means for dispensing includes a second lumen in said first catheter.

40. The apparatus according to claim 39, wherein:
said first lumen and said second lumen are coaxial.

41. The apparatus according to claim 39, wherein:
said means for dispensing includes said second lumen and a third lumen in said catheter, said second and third lumena being coupled at their distal ends.

42. The apparatus according to claim 39, wherein:
said first lumen is longer than said second lumen.

43. The apparatus according to claim 31, wherein:
said perforations each have substantially the same diameter.

44. The apparatus according to claim 31, wherein:
at least some of said perforations have different diameters.

45. The apparatus according to claim 31, wherein:
said perforations are evenly spaced.

46. The apparatus according to claim 31, wherein:
at least some of said perforations are variably spaced.

47. The apparatus according to claim 31, wherein;
at least some of said perforations are provided in spaced apart groups.

48. The apparatus according to claim 47, wherein:
said groups are evenly spaced.

49. The apparatus according to claim 47, wherein:
at least some of said groups are variably spaced.

50. The apparatus according to claim 47, wherein:
each of said groups contains the same number of perforations.

51. The apparatus according to claim 47, wherein:
at least some of said groups contain different numbers of perforations.

52. A method for treating the interior of a blood vessel, said method comprising:
delivering an apparatus into the blood vessel, the apparatus having
a first catheter having a proximal end and a distal end with a plurality of spaced apart perforations therebetween,
an occlusion device coupled to the distal end of the first catheter, and
deployment means for deploying said occlusion device;
deploying the occlusion device; and
dispensing a treating agent through the perforations in the first catheter proximal the occlusion device while maintaining the first catheter stationary.

53. The method according to claim 52, wherein:
the apparatus includes
a second catheter movable within the first catheter, the second catheter having at least one perforation adapted to align sequentially with the plurality of spaced apart perforations as the second catheter is moved through the first catheter, the second catheter adapted to receive a treating agent and dispense the treating agent through the at least one perforation, and said method further comprises
withdrawing the second catheter through the first catheter while dispensing treating fluid and maintaining the first catheter stationary.

54-130. (canceled)
